(19) United States
(12) Patent Application Publication
(10) Pub. No.: US 2022/0133929 A1
(43) Pub. Date: May 5, 2022

(54) TURF AND LARGE SURFACE DECONTAMINATION SYSTEM

(71) Applicant: Clean O3, LLC, Wichita, KS (US)

(72) Inventors: Daniel V. Drake, Wichita, KS (US); Buddy D. Gray, Dellrose, TN (US)

(73) Assignee: Clean O3, LLC, Wichita, KS (US)

(21) Appl. No.: 17/512,886

(22) Filed: Oct. 28, 2021

Related U.S. Application Data
(60) Provisional application No. 63/107,902, filed on Oct. 30, 2020.

(51) Int. Cl. A61L 2/18; A61L 2/22

(52) U.S. Cl. CPC A61L 2/183; A61L 2/22; A61L 2202/16; A61L 2202/15; A61L 2202/11

(57) ABSTRACT

A turf decontamination system includes a mobile device coupled to a host vehicle, an application system coupled to the mobile device, and a number of nozzles. The decontamination system includes a liquid and an ozone generator operative to create gaseous ozone. Each nozzle is configured to receive and combine the gaseous ozone and the liquid to create an ozone-liquid mixture. The ozone-liquid mixture is provided from an outlet according to a desired spray pattern to decontaminate the turf or surface beneath the system.

### CROSS-REFERENCE TO RELATED APPLICATIONS

This application claims priority from U.S. Provisional Patent Application Ser. No. 63/107,902, filed Oct. 30, 2020, which is hereby incorporated herein by reference in its entirety.

### BACKGROUND OF THE INVENTION

Viruses and bacteria are readily spread from person to person in numerous ways. For example, contaminated droplets from a sneeze or cough of an infected person may transfer the virus or bacteria through the air to be inhaled by a healthy person. Additionally, the virus or bacteria may be transferred to surfaces after contaminated droplets from a sneeze or cough come to rest on a surface, or after an infected person contacts the surface. Because viruses and bacteria can often survive for a period of time on the contaminated surface, contact with the contaminated surface by a healthy person may transfer the virus or bacteria to the healthy person, causing that person to become infected and continuing the transmission cycle as that person comes into contact with others and with multitudes of objects and surfaces during daily life.

One example of the ease at which a virus may spread, and the resulting dangers of widespread infection is the coronavirus disease 2019 (Covid-19) pandemic. Covid-19 has infected millions of people around the world and has killed hundreds of thousands. A large problem with containing such a pandemic includes the ability to disinfect athletic playing surfaces such as sports fields having artificial turf for playing surfaces. Turf fields include polymer or synthetic material. If one or more athletes playing on the turf is infected with Covid-19, contact with the turf has the potential to transfer the virus to the turf where it could potentially survive for a period of time that allows for the transfer to another athlete upon contact with the playing surface. If sports are to continue during a pandemic, there must be a reliable method for decontaminating the surfaces, fields, and courts on which athletes compete.

Oxidizing agents can be used to incapacitate or destroy pathogens. A strong oxidizing agent is ozone (O₃). Ozone occurs naturally in the environment when an electrical discharge, such as lighting, passes through air containing the gaseous form of oxygen (O₂). Pathogens harmful to humans include microorganisms, such as fungus, protozoan, bacteria, and viruses. Contact of an oxidizing agent with a pathogen can render the pathogen ineffective. Ozone is reactive for only a short time after it is generated, thus use of ozone as a disinfectant has limited residual harmful effects. Various embodiments of the present turf decontamination system recognize and address the foregoing considerations, and others, of prior art devices.

### SUMMARY

It should be appreciated that this Summary is provided to introduce a selection of concepts in a simplified form that are further described below in the Detailed Description. This Summary is not intended to be used to limit the scope of the claimed subject matter.

According to one aspect of the disclosure, a disinfecting system includes a mobile device, an application system coupled to the mobile device, and a number of nozzles. The mobile device is configured to be coupled to a host vehicle. The application system includes a liquid and an ozone generator operative to create gaseous ozone. Each nozzle is configured to receive the gaseous ozone and the liquid, and to combine the gaseous ozone and the liquid to create an ozone-liquid mixture. The nozzle is further configured to provide the ozone-liquid mixture from an outlet according to a desired spray pattern.

### DESCRIPTION OF THE DRAWINGS

The drawings constitute a part of this specification and include exemplary embodiments of the disclosed subject matter and illustrate various objects and features thereof.

FIG. 1 is a rear perspective view of an application system according to various embodiments disclosed herein.

FIG. 2 is a schematic of the application system according to various embodiments disclosed herein.

FIG. 3 is an enlarged view of the nozzle according to various embodiments disclosed herein.

FIG. 4 is a rear elevation view of the application system mounted to a trailer according to various embodiments disclosed herein.

FIG. 5 is a side elevation view of the application system mounted to a trailer according to various embodiments disclosed herein.

FIG. 6 is a front perspective view of the application system mounted to a trailer according to various embodiments disclosed herein.

FIG. 7 is a rear perspective view of the application system employing a covering according to various embodiments disclosed herein.

FIG. 8 is a perspective view of a drying agent of the application system for drying air prior to ozone generation according to alternative embodiments disclosed herein.

FIG. 9 is a perspective view of the application system showing a stowed boom according to alternative embodiments disclosed herein.

FIG. 10 is a rear perspective view of the application system showing a deployed boom according to alternative embodiments disclosed herein.

FIGS. 11-13 are various rear perspective views of the application system showing aspects of a deployed boom and boom lowering mechanism according to alternative embodiments disclosed herein.

### DETAILED DESCRIPTION OF THE INVENTION

As required, detailed aspects of the disclosed subject matter are disclosed herein; however, it is to be understood that the disclosed aspects are merely exemplary of the disclosed subject matter, which may be embodied in various forms. Therefore, specific structural and functional details disclosed herein are not to be interpreted as limiting, but merely as a basis for the claims and as a representative basis for teaching one skilled in the art how to variously employ the disclosed technology in virtually any appropriately detailed structure.

According to various embodiments described herein, an oxidizing agent application system mixes gaseous ozone with a liquid in a concentration sufficient to incapacitate and destroy Covid-19 and other pathogens on a surface. For purposes of this disclosure, the various implementations will be described in the context of eliminating Covid-19 and other pathogens from artificial turf. However, it should be appreciated that the various embodiments disclosed herein are equally applicable and effective at disinfecting any surface over which the system described herein may be used to treat. Examples include, but are not limited to any type of artificial or live/natural turf, indoor and outdoor gyms, auditoriums, courts, and athletic playing surfaces. If applied to non-porous surfaces such as a basketball or volleyball court, the applied solution would be allowed to disinfect for an appropriate time (e.g., 20-90 min) and then dried before use. The system described herein may even be used to disinfect conference rooms and other carpeted surfaces, allowing for appropriate drying time before use.

According to various embodiments, gaseous ozone is combined with the liquid in a nozzle by delivering the ozone to the nozzle at a higher pressure than the liquid, creating an ozone-liquid mixture. The ozone-liquid mixture exits the nozzle under pressure and is applied to the surface. An application device including an application system mounted to a mobile device, such as a trailer, applies the ozone-liquid mixture to a surface, such as a natural or artificial turf. The application system includes an oxidizing agent system, and a liquid system. The oxidizing agent system uses an ozone generator to create gaseous ozone and feeds the gaseous ozone to the nozzles. The liquid system moves the liquid to the nozzles that are attached in an array to a support structure located above the surface. The nozzles are where the gaseous ozone is combined with the liquid creating the ozone-liquid mixture with gaseous ozone dissolved or partially dissolved in the liquid. Dissolving or partially dissolving the ozone in a liquid, such as water, allows the ozone to remain in contact with the surface and any pathogens thereon, oxidizing the pathogens, and rendering any pathogens thereon ineffective.

A surfactant or wetting agent may be added to the liquid and gaseous ozone to create an ozone-surfactant-liquid material. The addition of a surfactant to the liquid decreases the tension of the resulting mixture thereby increasing the amount of the ozone-liquid mixture in contact with the surface and the ozone in contact with any pathogens thereon. A covering may be connected to the support structure to direct the ozone-liquid mixture and any undissolved gaseous ozone to the surface, and aid in retaining any undissolved gaseous ozone with the liquid on the surface to replace the ozone that is consumed in the oxidative process.

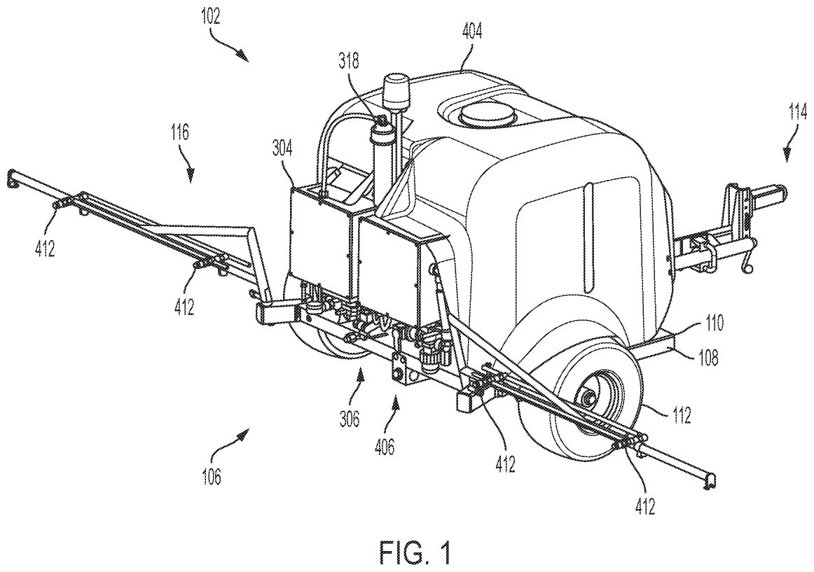

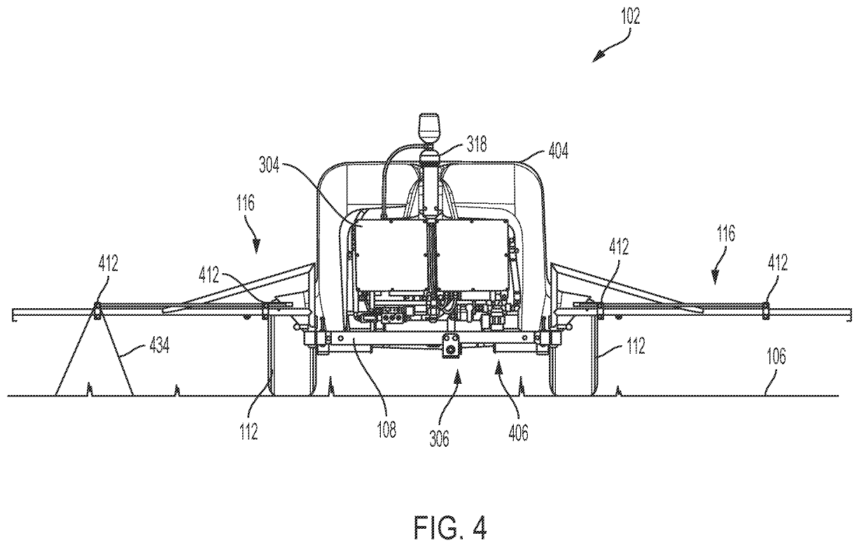

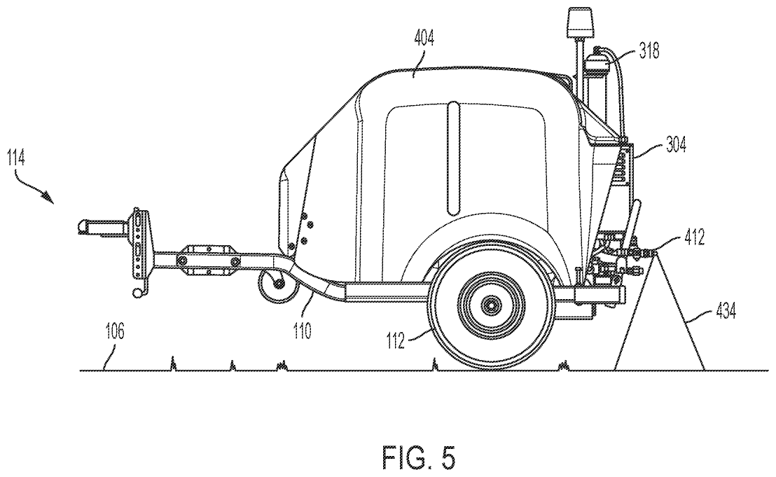

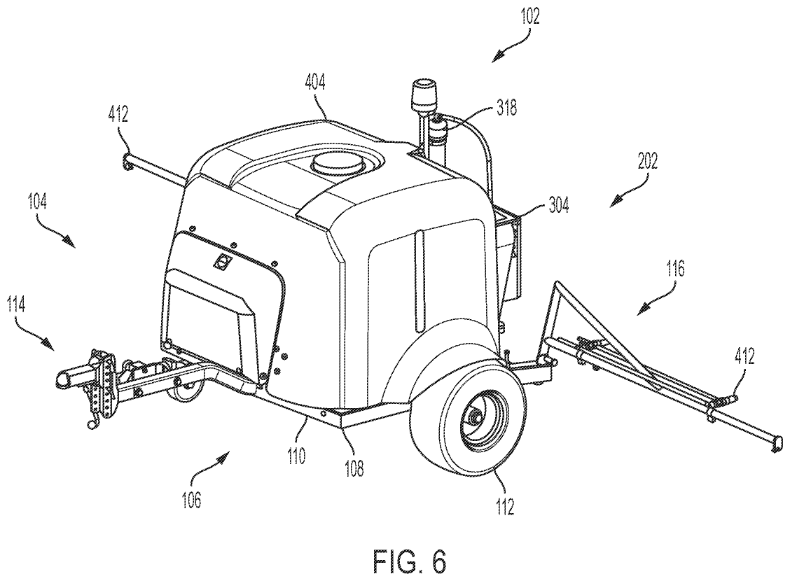

Referring now to the drawings, an application device 102 including an application system 202 mounted to a mobile device 104 for applying an oxidizing agent to a surface 106 is shown in FIGS. 1-13. The mobile device 104 may be any device movable across a surface. In an embodiment, the application system 202 is mounted to a self-powered vehicle, such as a truck. In another embodiment of the disclosed subject matter, the mobile device 104 is a trailer 108. The trailer 108 includes a frame 110 with wheels 112 allowing the trailer 108 to be moved about the surface 106. The frame 110 includes a connecting member 114 for connecting the trailer 108 to a vehicle (not shown) for moving the trailer 108 about the surface 106. In an embodiment, the connecting member 114 is a receiver for a ball-type or pin-type hitch. According to various embodiments, the surface 106 is turf of a natural or artificial nature.

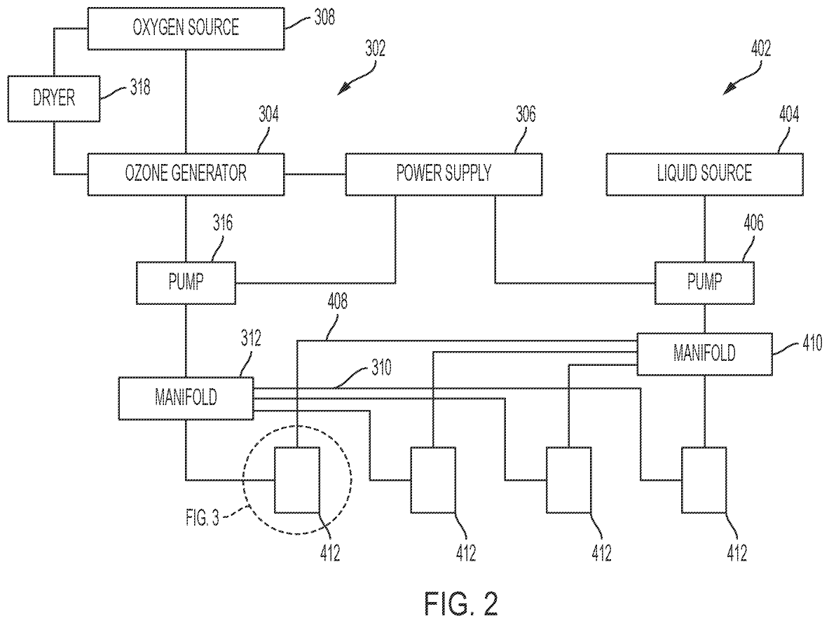

Referring to FIG. 2, the application system 202 includes an oxidizing agent system 302 and a liquid system 402, energized by a power supply 306. The ozone generator 304 may be any ozone generator suitable for mounting on a mobile device 104, including the KH-CT5G ozone generating unit manufactured by DGOzone Ltd. of Shanghai, China. In an embodiment, the power supply 306 is a battery with an inverter providing AC power. In an embodiment, the power supply 306 includes electricity generated by a combustion engine connected to an electrical generator providing AC power. According to another embodiment, the power supply 306 is located on the host vehicle and coupled to the application system 202 via one or more power cables. In this implementation, the vehicle pulling the mobile device 104 connects to the application system 202 via a power connector, similar to a conventional brake light and turn signal connector on a typical trailer. However, depending on the power requirements for the application system 202, any type of suitable power connector and corresponding electrical cables/wires may be used. According to one embodiment, the application system 202 includes a single point switch that is operative to turn the application system 202 on and off. The switch may be located on or in the application device 102, or may alternatively or additionally be positioned within the host vehicle and electrically coupled to the application device 102 via the connectors and cables that couple the application device 102 to the host vehicle when the host provides the power supply 306.

In an embodiment, the liquid system 402 includes a liquid source 404 connected to nozzles 412. In an embodiment, the liquid source 404 is a tank 414 containing water. A pump 406, operably connected to the power supply 306, moves the liquid 405 from the liquid source 404 to the nozzles 412 as a pressurized liquid by conduits 408 and a manifold 410. The nozzles 412 are arranged in an array to apply the liquid 405 to the surface 106 in a consistent manner. In an embodiment, the nozzles 412 are spaced along a support structure, such as a boom 116, at an end of the frame 110.

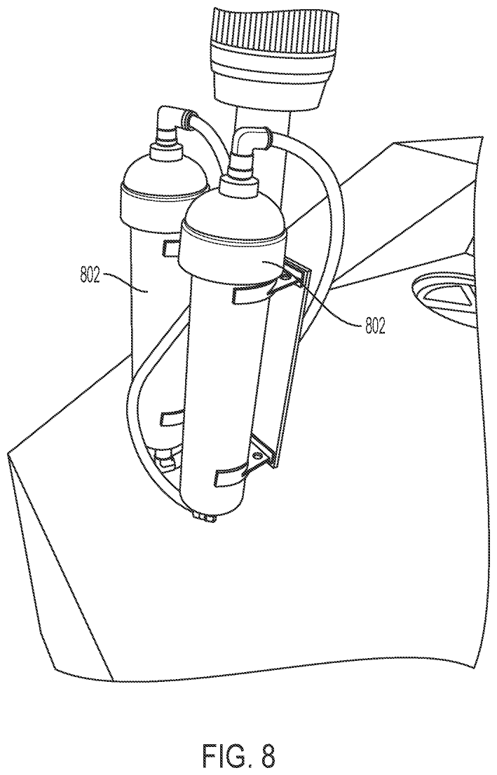

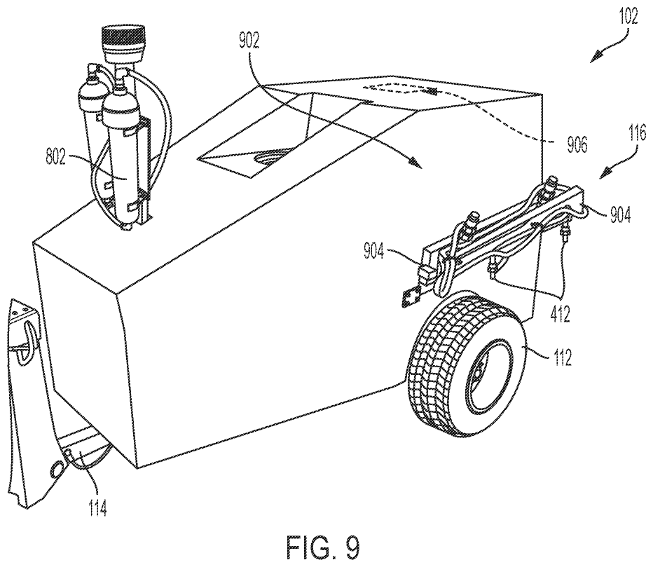

According to various embodiments, the oxygen source 308 is ambient air from the environment containing the gaseous form of oxygen that is passed through a dryer 318 and then fed to the ozone generator 304 by a pump operably connected to the power supply 306. In another embodiment, the oxygen source 308 is the gaseous form of oxygen in the form of either liquefied oxygen or compressed oxygen gas that is bled to the ozone generator 304 by a regulator and valve. Any type of dryer 318 may be used to remove moisture from the air being provided to the ozone generator 304. According to one embodiment, shown in FIGS. 8 and 9, the dryer 318 includes one or more desiccant vessels 802, which contain desiccant beads or silica gel beads that absorb moisture from the air. In the example shown in FIGS. 8 and 9, two desiccant vessels 802 are used, although any desired number of desiccant vessels 802 of any volume may be used without departing from the scope of this disclosure.

The oxygen containing material from the oxygen source 308 passes through the ozone generator 304, and the ozone generator 304 creates gaseous ozone 314 by passing an electrical current through material. The gaseous ozone 314 is transferred from the ozone generator 304 to the manifold 312, then to each nozzle 412 where the gaseous ozone 314 combines with the liquid 405. In an embodiment, the gaseous ozone 314 is transferred by a pump 316 at a pressure of about 60 pound per square inch (psi). The ozone generator 304 is operated by a user using conventional control mechanisms.

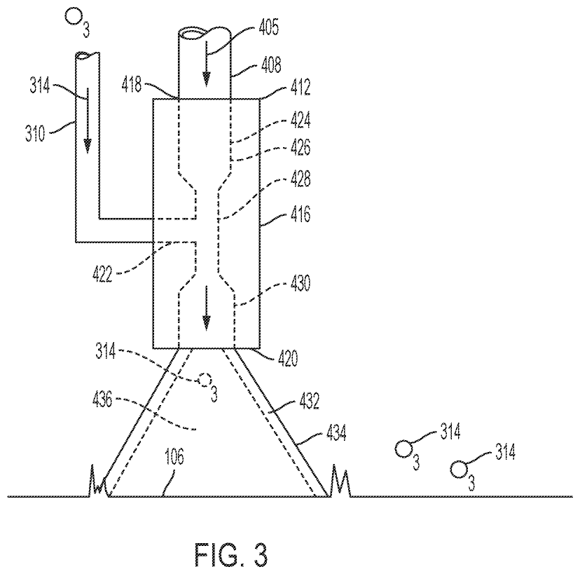

Referring to FIG. 3, the nozzles 412 include a body 416 defining a liquid flow passage 242 with an inlet 418 at one end and an outlet 420 at an opposite end. The liquid flow passage 424 has a wide diameter upper portion 426 immediately above a smaller diameter middle portion 428. A port 422 located between the inlet 418 and outlet 420 extends from the exterior of the nozzle 412 through the body 416 communicating with the middle portion 428. Pressurized liquid 405 at a pressure of about 20 psi enters the liquid passage 424 through the inlet 418 via the conduit 408.

Gaseous ozone 314 at about 60 psi enters the liquid passage 424 through the port 422 via the conduit 310. The venturi effect occurring at the middle portion 428 draws gaseous ozone 314 into the liquid 405 stream as it passes through the constriction mixing the gaseous ozone 314 with the liquid 405. A portion of the gaseous ozone 314 dissolves in the liquid 405 and the ozone-liquid mixture 432 exits the nozzle 412 through the outlet 420 and is applied to the surface 106. In an embodiment, the gaseous ozone 314 dissolved in the liquid 405 is about 0.6 milligrams per liter of liquid 405. The liquid system 402 is operated by a user using conventional control mechanisms.

By ozonation of the liquid 405, the ozone-liquid mixture 432 can be applied to the surface 106 and remain in contact with the surface 106. The ozone-liquid mixture 432 holds the dissolved gaseous ozone 314 in contact with the surface 106 and any pathogens thereon as the oxidizing agent disinfects the surface 106 by rendering any pathogens thereon ineffective. As oxidization of the pathogens occurs, the ozone reverts back to the gaseous form of oxygen leaving no harmful residue behind.

In an embodiment, the ozone-liquid mixture 432 exits the nozzle 412 in a conical spray pattern 434 as it is applied to the surface 106. In an embodiment, the conical spray pattern 434 is a solid cone of the ozone-liquid mixture 432. Applying the ozone-liquid mixture 432 to the surface 106 as the nozzles 112 move across the surface coats the surface 106 with the ozone-liquid mixture 432. In an embodiment, approximately 0.00162894 gallons of ozone-liquid mixture 432 is applied to each square foot of the surface 106.

In an embodiment, the conical spray pattern 434 is a hollow cone of the ozone-liquid mixture 432 with an open interior area 436. Within the interior area 136, undissolved gaseous ozone 314 is carried down to the surface 106. In another embodiment, the ozone-liquid mixture 432 exits the nozzle 412 in a fan spray pattern as it is applied to the surface 106.

Gaseous ozone 314 that either comes out of the ozone-liquid mixture 432 or that is not dissolved in the liquid 405 in the nozzle 412 moves along with the ozone-liquid mixture 432 toward the surface 106 coating the surface 106 with a cloud of gaseous ozone 314. In an alternative embodiment, the liquid 405 is not used and gaseous ozone is 314 is applied directly to the turf. In yet another embodiment, the ozone-liquid mixture 432 is applied through the nozzles 412 as described above, however, in addition to the ozone-liquid mixture 432, gaseous ozone 314 is additionally applied to expedite the disinfecting process. In this embodiment, a portion of the gaseous ozone 314 created from the ozone generator 304 may be directed to the nozzles 412, or may be directed to separate nozzles dedicated to distributing gaseous ozone. A separate ozone generator 304 may alternatively be used to generate gaseous ozone 314 to be applied directly to the turf.

The nozzles 412 are spaced apart from each other and positioned at a height above the surface 106 to achieve the desired spray coverage and/or overlap of spray on the surface 106. In an embodiment, the nozzles 412 are spaced and positioned to achieve an overlap of about five inches of spray coverage. As will be described in greater detail below, the boom and nozzles may be configured to cover a spray width of at least two feet during sanitation.

In an embodiment, a surfactant or wetting agent is added to the liquid 405 prior to the mixing of the gaseous ozone 314 with the liquid 405 in the nozzle 412 forming a surfactant-liquid mixture. The surfactant-liquid mixture enters the inlet 418 and gaseous ozone 314 is dissolved in the surfactant-liquid mixture, as described above with respect to the liquid 415, forming an ozone-surfactant-liquid mixture. The ozone-surfactant-liquid mixture exits the nozzle 412 through the outlet 420, as described above, and is applied to the surface 106. The addition of a surfactant to the liquid 405 decreases the tension of the liquid 405 increasing the amount of the ozone-liquid mixture in contact with the surface 106, and the ozone in contact with any pathogens on the surface 106. In an embodiment, the surfactant is the surfactant sold under the trademark BARDACAC® LF-80 from Lonza, Inc. of Allendale, N.J.

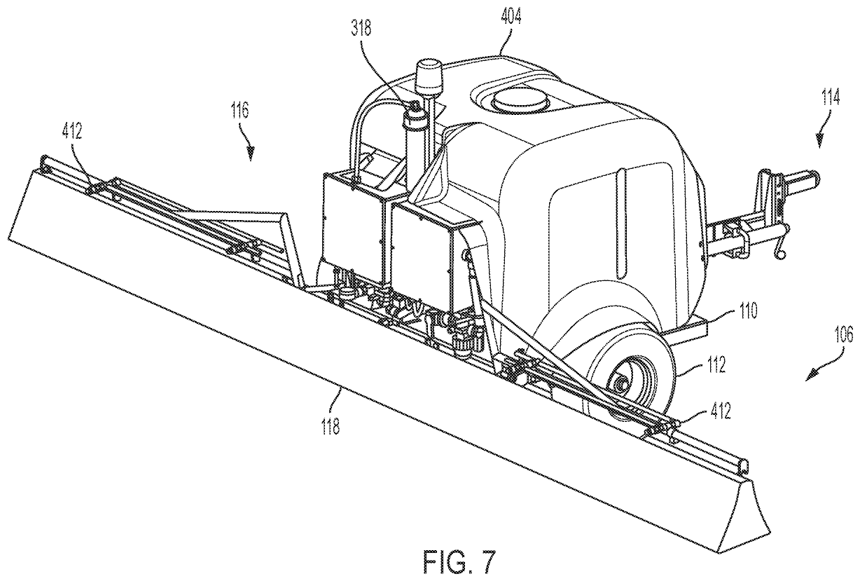

In an embodiment, a covering 118 extends downward and outward away from the nozzles 412 directing the ozone-liquid mixture 432 and cloud of gaseous ozone 314 downward after it is emitted from the nozzle 412 to minimize dispersion and dilution of the gaseous ozone 314 (FIG. 7). Directing the gaseous ozone 314 to the surface 106 allows the gaseous ozone 314 to be present in the air close to the ozone-liquid mixture 432 allowing the gaseous ozone 314 to diffuse into the ozone-liquid mixture 432 replacing the ozone that is consumed in the oxidative process. The covering 118 also confines the ozone-liquid mixture 432 and the undissolved gaseous ozone 314 close to the surface 106. The covering 118 may be manufactured from a rigid material, such as plastic, or may be manufacture from a flexible material, such as a water resistant fabric.

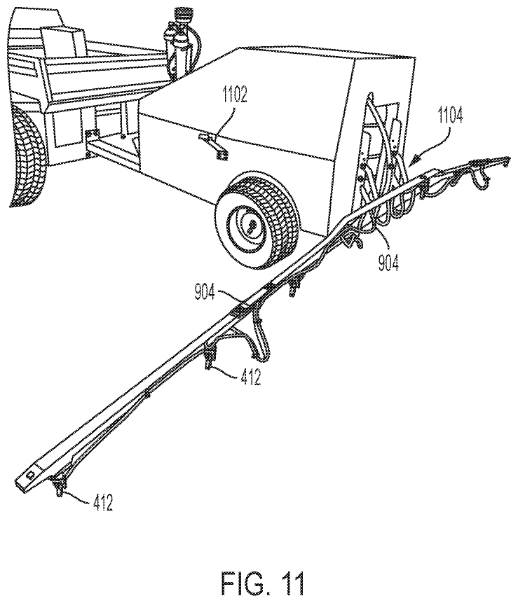

Turning now to FIG. 9, aspects of the boom 116 will be discussed. According to one embodiment, the boom 116 may be selectively reconfigured between a stowed configuration and an operational configuration. The stowed configuration is shown in FIG. 9. In the stowed configuration, the boom 116 is folded around pivots 904 and secured against the sides of the application device 102. According to one embodiment, the application device 102 may include a hood 902 that covers and protects the sanitizing components of the application system 202. The boom 116 may rest against and be secured to the hood 902 when positioned in the stowed configuration using a bracket 1102 (shown in FIGS. 11-13).

The pivots 904 may include hinges, pins, flexible rubber or polymer sleeves, or any combination thereof. Any mechanism that allows for adjacent sequential segments to rotate with respect to one another to fold and unfold the boom 116 may be utilized without departing from the scope of this application. According to various embodiments, the boom 116 may be configured to flex or break away when extended and upon contact with a structure or obstacle. In this manner, damage to the boom 116 or corresponding application device 102 may be prevented. This break away feature may be implemented via the pivots 904. The pivots 904 may be constructed to rotate the adjacent boom sections with respect to one another in response to a predetermined force applied to one or both of the boom sections.

FIG. 9 shows a safety feature of the application device 102. Specifically, according to one embodiment, the application device 102 may include one or more safety lights 906 that illuminate to provide notification of a decontamination process that is in progress. The one or more safety lights 906 may automatically illuminate upon activation of the application system 202 and deactivate upon completion, or may be manually activated and deactivated. Any number and type of safety lights 906 of any color and positioning may be utilized without departing from the scope of this disclosure.

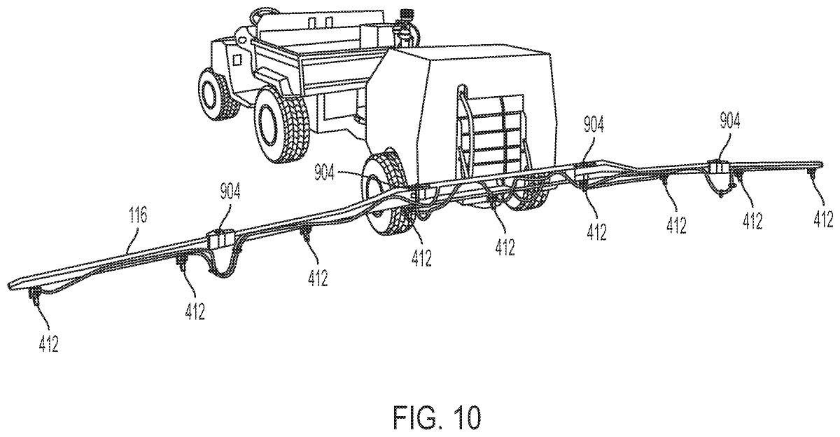

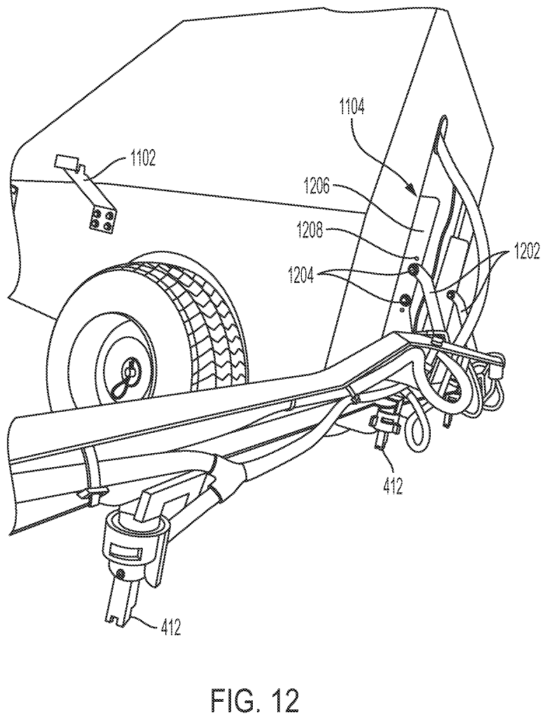

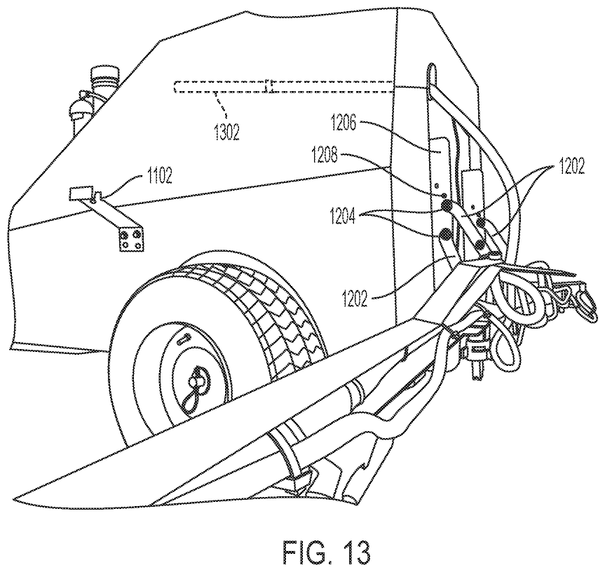

FIG. 10 shows a rear perspective view of the application device 102 with the boom 116 extended in the operational configuration. In this configuration, the boom segments are rotated around the pivots 904 from the stowed configuration and extended to a substantially linear arrangement such that the boom 116 extends to a maximum or desired width for treating the applicable turf. The pivots 904 may be locked with pins or any other suitable methods to maintain the boom 116 in the operational configuration. Moreover, after the boom 116 is extended, the boom 116 is lowered to a desired height over the turf. According to one embodiment, the lowering and raising of the boom 116 to transition the boom 116 between stowed and operational configurations is facilitated using a height adjustment mechanism 1104, as seen in FIG. 11. The height adjustment mechanism 1104 is more clearly seen in FIGS. 12 and 13.

According to one embodiment, the height adjustment mechanism 1104 includes one or more anchors 1206 that are secured to the application device 102. One or more pivot arms 1202 connect the boom 116 to the anchors 1206 via adjustment pivots 1204. A height securement mechanism 1208 may be used to secure the boom 116 in the raised, lowered, or any intermediate positions. The height securement mechanism 1208 may include one or more pins and corresponding pin apertures positioned according to desired boom heights 116. As the boom 116 is rotated between stowed and fully lowered operational configurations, the pin apertures within the pivot arms 1202 may traverse the anchors or other structure and align with corresponding pin apertures. Securing a pin within the aligned pin apertures will secure the boom 116 at a corresponding height. The height adjustment mechanism 1208 of the various drawings show boom 116 positions of fully raised for stowage and fully lowered for operation. It should be understood that any number of intermediate height locations may be provided as desired, depending on the specific implementation of the application device 102. As can be seen in FIGS. 9 and 11, the boom 116 may be configured to be positioned above the wheels 112 when in the stowed configuration, and lowered to a position below the tops of the wheels 112 when in the operational configuration.

FIG. 13 shows another embodiment in which the application device 102 includes an optional disinfecting wand 1302 in a stowed configuration. In this embodiment, the disinfecting wand 1302 may be unstowed and activated to redirect all or a portion of the ozone-liquid mixture 432 from the nozzles 412 to the disinfecting wand 1302. The user may then direct the wand to specific areas or objects for disinfecting. This implementation is useful for smaller areas that require disinfecting but are inaccessible by the application device 102 without the wand.

It should be appreciated from the above disclosure that the application device 102 and corresponding application system 202 described herein may be utilized to eliminate Covid-19 and other pathogens from natural and artificial turf and other large areas. The tank 414 of liquid may be sized to provide for treatment of at least an entire football field without stopping at approximately 3-5 mph. Using the above systems, test results have shown elimination of Covid-19 from a turf field at a single pass at 3 mph at a 99.52% effectiveness rate.

It is to be understood that while certain aspects of the disclosed subject matter have been shown and described, the disclosed subject matter is not limited thereto and encompasses various other embodiments and aspects.

What is claimed is:

1. A disinfecting system, comprising:
a mobile device configured to be coupled to a host vehicle; and
an application system coupled to the mobile device and comprising:
an ozone generator operative to create gaseous ozone;
a liquid; and
a plurality of nozzles, each nozzle configured to receive the gaseous ozone and the liquid, and to combine the gaseous ozone and the liquid to create an ozone-liquid mixture, the nozzle further configured to provide the ozone-liquid mixture from an outlet according to a desired spray pattern.

2. The disinfecting system of claim 1, wherein the plurality of nozzles are positioned on a boom, the boom being selectively configurable between a stowed configuration and an operational configuration.

3. The disinfecting system of claim 2, wherein the boom further comprises a plurality of pivots configured to fold the boom to the stowed configuration and unfold the boom to the operational configuration.

4. The disinfecting system of claim 3, further comprising a height adjustment mechanism configured to selectively raise and lower the boom to vary a height over a treatment surface.

5. The disinfecting system of claim 1, wherein the application system further comprises one or more desiccant vessels, each desiccant vessel fluidly coupled to the ozone generator and configured to remove moisture from ambient air prior to routing the ambient air to the ozone generator.

6. The disinfecting system of claim 1, wherein the application system further comprises a disinfecting wand, wherein the application system is operative to selectively route at least a portion of the ozone-liquid mixture through the disinfecting wand.
